(19) United States
(12) Patent Application Publication
(10) Pub. No.: US 2022/0096789 A1
(43) Pub. Date: Mar. 31, 2022

(54) Coaxial Multi-Lumen Catheter

(71) Applicant: Bard Access Systems, Inc., Salt Lake City, UT (US)

(72) Inventor: Gidon Ofek, Millcreek, UT (US)

(21) Appl. No.: 17/487,471

(22) Filed: Sep. 28, 2021

Related U.S. Application Data
(60) Provisional application No. 63/085,065, filed on Sep. 29, 2020.

(51) Int. Cl. A61M 25/00

(52) U.S. Cl. CPC A61M 25/003; A61M 25/0075; A61M 2210/12; A61M 2205/0216; A61M 2025/0039

(57) ABSTRACT

A multi-lumen catheter with coaxial geometry that provides for a tube within a tube design. The coaxial multi-lumen catheter is designed to minimize or preclude reflux incidents where liquids from one lumen can mix with the liquids from another lumen, or flow retrograde in the other lumen. The coaxial catheter geometry can provide for a secondary support from an outer lumen during infusion pressurization. For example, a fluid pressure from an outer lumen can support an inner lumen, preventing radial expansion. Alternatively a wall of the inner lumen can impinge on an outer wall to prevent further radial expansion. An outer lumen can terminate proximally of an inner lumen to provide different infusion sites along a patient's vasculature.

### PRIORITY

This application claims the benefit of priority to U.S. Provisional Application No. 63/085,065, filed Sep. 29, 2020, which is incorporated by reference in its entirety into this application.

### BACKGROUND

A variety of multi-lumen catheters can be used by clinicians to provide fluids, blood or medications to or from a patient. Conventional catheters can include one or more lumen extending parallel and adjacent to each other through a body of the catheter. A longitudinal axis of a first lumen is laterally or transversely off-set from a longitudinal axis of a second lumen. The cross-sectional shape of these lumen can include circular, ellipsoidal, semi-circular, semi-ellipsoidal, or “D-shaped”, or various polygonal cross-sectional shapes. The cross-sectional shape of the lumen can have an effect on the fluid flow dynamics through the lumen. Circular lumen can provide preferable fluid dynamics but adjacent circular lumen can provide a relatively large outer surface profile. “D-shaped” lumen designs can provide a reduced outer profile however occurrences of an “opposite lumen reflux” can occur where flow from a first lumen can affect the flow from an adjacent lumen. This in turn can affect infusion rates, and can be detrimental to a patient. Disclosed herein is a coaxial multi-lumen catheter that mitigates or precludes incidents of the opposite lumen reflux and provides for safer infusions.

### SUMMARY OF THE INVENTION

Briefly summarized, disclosed herein is a multi-lumen catheter with coaxial geometry. The coaxial catheter geometry provides for a tube within a tube design. The coaxial multi-lumen catheter minimizes or precludes reflux incidents where liquid medications from one lumen can mix with liquids from another lumen. The coaxial catheter geometry provides for a secondary support from an outer lumen during infusion pressurization.

Disclosed herein is a multi-lumen catheter including, an outer wall defining an outer lumen having an outer lumen diameter and extending along a central longitudinal axis, and an inner wall defining an inner lumen extending co-axially with the outer lumen and transitionable between a first position and a second position, the inner lumen in the first position having a first inner lumen diameter, and the inner lumen in the second position having a second inner lumen diameter larger than the first inner lumen diameter.

In some embodiments, the inner wall is elastically deformable radially outwards from the first position to the second position. In some embodiments, one of the first inner diameter lumen or the second inner lumen diameter is equal to or less than the outer lumen diameter. In some embodiments, an outer surface of the inner wall contacts the inner surface of the outer wall when the inner lumen is in the second position. In some embodiments, the outer wall is formed of a first material, and the inner wall is formed of a second material, different from the first material. In some embodiments, the second material is more compliant, provides a lower durometer, is softer, or provides a lower modulus of elasticity relative to the first material.

In some embodiments, a first material of the outer wall includes a plastic, polymer, urethane, or polyurethane, and a second material of the inner wall includes one of silicone rubber, rubber, or elastomer. In some embodiments, the inner wall in second position impinges on a surface of the outer wall, the outer wall preventing further radial expansion of the inner wall. In some embodiments, the outer wall defines a first thickness and the inner wall defines a second thickness, less than the first thickness. In some embodiments, the outer wall defines a first thickness and the inner wall defines a second thickness, one or both of the first thickness and the second thickness defines a uniform thickness along a longitudinal length of the catheter.

In some embodiments, the outer wall defines a first thickness and the inner wall defines a second thickness, one or both of the first thickness and the second thickness defines a change in thickness along a longitudinal length of the catheter. In some embodiments, one of the first inner lumen diameter or the outer lumen diameter defines a uniform diameter along a longitudinal length of the catheter. In some embodiments, one of the first inner lumen diameter or the outer lumen diameter defines a change diameter along a longitudinal length of the catheter. In some embodiments, the inner lumen defines a first longitudinal length, and the outer lumen defines a second longitudinal length, the second longitudinal length being less than the first longitudinal length.

In some embodiments, a distal tip of the outer lumen is configured to terminate in an upper arm portion of a patient, and a distal tip of the inner lumen is configured to terminate in a superior vena cava. In some embodiments, the multi-lumen catheter further includes a bifurcation hub, a first extension leg in fluid communication with the inner lumen, and a second extension leg in fluid communication with the outer lumen. In some embodiments, one of the inner lumen or the outer lumen includes a closed distal tip and a valve.

Also disclosed is a catheter system including, a first wall defining a first lumen extending along a central longitudinal axis, a second wall defining a second lumen extending co-axially with the first lumen along the central longitudinal axis, the first wall encircling the second wall, a third wall defining a third lumen extending co-axially with the first lumen and the second lumen along the central longitudinal axis, the first wall and the second wall encircling the third wall, one of the second wall or the third wall is elastically deformable radially outwards.

In some embodiments, one or both of the second lumen and the third lumen define a lumen diameter is equal to or less than a lumen diameter of the first lumen. In some embodiments, the third wall is configured to elastically deform radially outwards until an outer surface of the third wall impinges on the inner surface of the second wall. In some embodiments, the second wall is configured to elastically deform radially outwards until an outer surface of the second wall impinges on the inner surface of the first wall. In some embodiments, the first wall is configured to prevent radial outward expansion.

In some embodiments, the first wall is formed of a first material, and one or both of the second wall and the third wall is formed of a second material, different from the first material. In some embodiments, the third wall is formed of a third material different from the first material and the second material. In some embodiments, one or both of the second material and the third material is more compliant, provides a lower durometer, is softer, or provides a lower modulus of elasticity relative to the first material. In some embodiments, the first material includes a plastic, polymer, urethane, or polyurethane, and one or both of the second material and the third material includes one of silicone rubber, rubber, or elastomer.

In some embodiments, the first wall, second wall and third wall define the same wall thickness. In some embodiments, one of the first wall, second wall, or third wall define a different wall thickness. In some embodiments, the first wall, second wall and third wall define a uniform wall thickness along a longitudinal length of the catheter. In some embodiments, one of the first wall, second wall, or third wall define different wall thicknesses along a longitudinal length of the catheter. In some embodiments, the first lumen, second lumen and third lumen define a uniform lumen diameter along a longitudinal length of the catheter.

In some embodiments, one of the first lumen, second lumen, or third lumen define different lumen diameters along a longitudinal length of the catheter. In some embodiments, the first lumen, second lumen and third lumen define the same longitudinal length between a proximal end of the catheter and a distal tip of the catheter. In some embodiments, one of the second lumen or third lumen terminate at a point proximal of a distal tip of the catheter.

Also disclosed is a method of providing vascular access including, placing a distal tip of a catheter within a vasculature of a patient, the catheter including a first wall defining a first lumen and a second wall defining a second lumen extending co-axially with the first lumen, the second wall encircling the first wall, providing a fluid flow to the first lumen, and elastically deforming the first wall radially outwards.

In some embodiments, the method further includes providing a second fluid flow to the second lumen to provide a radially inward pressure. In some embodiments, the method further includes impinging an outer surface of the first wall on an inner surface of the second wall, the second wall preventing further radial expansion. In some embodiments, the method further includes a third wall defining a third lumen extending co-axially with the first lumen and the second lumen, the third wall encircling the second wall.

In some embodiments, the method further includes impinging an outer surface of the second wall on an inner surface of the third wall, the third wall preventing further radial expansion. In some embodiments, the method further includes terminating the first lumen at a superior vena cava and terminating the second lumen at an upper arm of the patient. These and other features of the concepts provided herein will become more apparent to those of skill in the art in view of the accompanying drawings and following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A provides a lateral cross-section view of a dual-lumen co-axial catheter, in accordance with some embodiments.

FIG. 1B provides a longitudinal cross-section view of a dual-lumen co-axial catheter, in accordance with some embodiments.

FIG. 2A provides a lateral cross-section view of a dual-lumen co-axial catheter, in accordance with some embodiments.

FIG. 2B provides a longitudinal cross-section view of a dual-lumen co-axial catheter, in accordance with some embodiments.

FIG. 3A provides a lateral cross-section view of a triple-lumen co-axial catheter, in accordance with some embodiments.

FIG. 3B provides a longitudinal cross-section view of a triple-lumen co-axial catheter, in accordance with some embodiments.

FIG. 4A provides a lateral cross-section view of proximal end of a dual-lumen co-axial catheter, in accordance with some embodiments.

FIG. 4B provides a longitudinal cross-section view of proximal end of a dual-lumen co-axial catheter, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

In the following description, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

With respect to “proximal,” a “proximal portion” or a “proximal-end portion” of, for example, a catheter disclosed herein includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter disclosed herein includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

To assist in the description of embodiments described herein, a longitudinal axis extends substantially parallel to an axis of the catheter. A lateral axis extends normal to the longitudinal axis, and a transverse axis extends normal to both the longitudinal and lateral axes. As used herein, a horizontal plane extends along the lateral and longitudinal axes. A vertical plane extends normal to the horizontal plane.

Unless defined otherwise, all technical and scientific terms used herein have the same meaning as commonly understood by those of ordinary skill in the art.

Embodiments disclosed herein are directed to a multi-lumen catheter with coaxial geometry that provides for a “tube-within-a-tube” design. The coaxial multi-lumen catheter mitigates or precludes opposite lumen reflux incidents where fluid flow from a first lumen can detrimentally affect the fluid flow from a second lumen. Advantageously, the coaxial catheter geometry can further provide for a secondary support to the inner lumen, either from a fluid pressure within the outer lumen, or directly from a wall of the outer lumen, during infusion. In an embodiment, an outer lumen can advantageously terminate proximally of a distal tip of the catheter 100 to provide different infusion sites within a patient's vasculature. For example, the outer lumen may terminate at an upper arm target location, while a central (i.e., inner) lumen may terminate at a superior vena cava (“SVC”) target location. Further, a relatively shorter longitudinal length of a lumen may allow for higher infusion rates.

In an embodiment, the coaxial catheter geometry can further provide for a “double wall” support for an inner lumen. For example, expansion of an inner lumen during a high pressure infusion (“power infusion”) can cause the inner lumen to radially expand. In an embodiment, a fluid pressure within an outer lumen can mitigate radial expansion of the inner lumen. In an embodiment, a wall of the inner lumen can radially expand and impinge on the wall of the outer lumen. The wall of the outer lumen may prevent further radial expansion of the inner lumen and provide for a secondary support during infusion. In an embodiment, the inner lumen may have thinner wall since it can be supported by the outer lumen wall during infusion. In an embodiment, the inner lumen can include a softer, more compliant material relative to the wall of the outer lumen since it can be supported by the outer lumen wall during infusion. Advantageously, the softer or thinner wall of the inner lumen can provide for a smaller overall cross-sectional size of the catheter. These and additional embodiments are described in more detail herein.

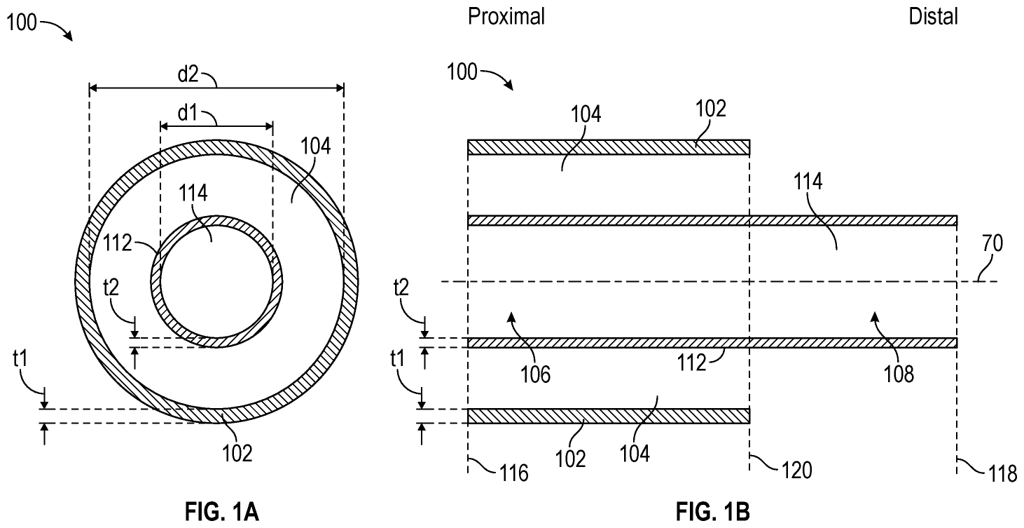

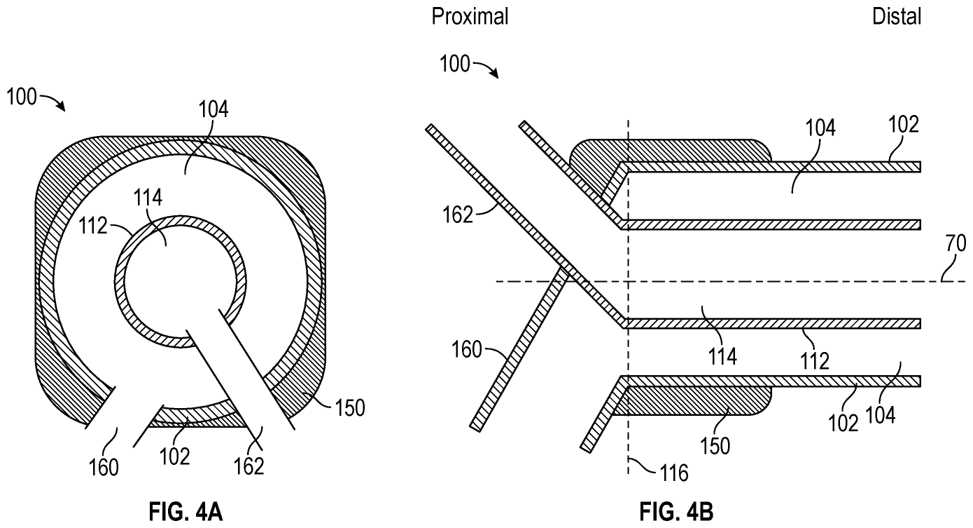

Referring to FIGS. 1A-1B, a dual-lumen coaxial catheter (“catheter”) 100 is provided. FIG. 1A shows a lateral cross-sectional view. FIG. 1B shows a longitudinal cross-sectional view. In an embodiment, the catheter 100 can extend along a central longitudinal axis 70 and include an outer wall 102 defining an outer lumen 104, and an inner wall 112 defining an inner lumen 114. The cross sectional views illustrate how the coaxial catheter geometry uses a tube within a tube design that provides numerous advantages discussed above. As shown in FIG. 1B, the catheter 100 can extend from a proximal end 116 to a distal end 118. In an embodiment, a proximal end 116 of the catheter 100 can be coupled to a hub 150, and can include one or more extension legs 160, 162 (FIGS. 4A-4B). In an embodiment, each extension leg 160, 162 can be in fluid communication with a lumen of the catheter 100, as described in more detail herein.

In an embodiment, both the outer lumen 104 and the inner lumen 114 of the catheter 100 can extend from a proximal end 116 to a distal tip 118. In an embodiment, a distal tip 120 of the outer lumen 104 can terminate at a point proximal of the distal tip 118 of the catheter 100. For example, the distal tip 120 of the outer lumen 104 can terminate at a point in an upper arm of a patient, while the distal tip 118 of the catheter 100, i.e. a distal tip of the inner lumen 114, terminates at the SVC 118. The relatively shorter lumen of the outer lumen 104 may advantageously allow for higher infusion rates to be provided.

Since a pressure drop along a tube is proportional to its length, pressures exerted on the inner lumen 114 at a distal portion 108 of the catheter 100 are lower than pressures at a proximal portion 106 of the catheter 100. For example, a region of the inner lumen 114, between the proximal end 116 and the distal tip 120 of the outer lumen 104 can be considered a relatively higher pressure region 106. Similarly, a region of the inner lumen 114, between the distal tip 120 of the outer lumen 104 and the distal tip 118 of the catheter 100 can be considered a relatively low pressure region 108.

In an embodiment, the wall 112 of the inner lumen 114 can be configured to support a pressure of the relatively low pressure region 108, since this region of the inner lumen 114 is unsupported. However, the wall 112 of the inner lumen 114 can be thinner, or weaker, than would otherwise be required to support the relatively high pressure region 106 since the proximal portion of the inner lumen 114 is further supported by the outer lumen 104, or an outer lumen wall 102. For example, the relatively high pressure region 106 of the inner lumen 114 can be supported by one or both of a fluid pressure within the outer lumen 104, and the wall 102 of the outer lumen 104, which can resist radial expansion, or further radial expansion, of the inner lumen 114.

In an embodiment, a fluid pressure within the outer lumen 104 can exert a radially inward pressure on the inner lumen wall 112 and can prevent, or mitigate radial expansion of the inner lumen wall 112. In an embodiment, the inner lumen wall 112 can be elastically deformable between a first position, defining a first inner lumen diameter, and a second position, defining a second inner lumen diameter. The second inner lumen diameter can be larger than the first inner lumen diameter. In an embodiment, the fluid pressure within the outer lumen 104 can maintain the inner lumen diameter at one of the first inner lumen diameter or the second inner lumen diameter. In an embodiment, the inner lumen wall 112 can elastically deform and an outer surface of the inner lumen wall can impinge on an inner surface of the outer lumen wall 102. The outer lumen wall 102 can prevent any further radial expansion of the inner lumen 114. Advantageously, the inner lumen 114 can define a thinner wall and smaller overall cross-sectional profile while still being capable of sustaining high infusion rates.

In an embodiment, a wall 102 of the outer lumen 104 can define a first thickness (t1), and a wall 112 of the inner lumen 114 can define a second wall thickness (t2), the first wall thickness (t1) can be greater than a second wall thickness (t2). In an embodiment, one or both of the inner lumen wall 102 and the outer lumen wall 112 can define a uniform wall thickness between a proximal end 116 and a distal tip 118 of the catheter 100. In an embodiment, an inner lumen wall 112 and an outer lumen wall 114 can be formed of the same material. In an embodiment, an inner lumen wall 112 and an outer lumen wall 114 can be formed of different materials, displaying different mechanical properties. For example, an inner lumen wall 112 can be formed of a softer material, a more compliant material, or a material having a lower modulus of elasticity (i.e. easier to elastically deform), or combinations thereof, relative to an outer lumen wall 102.

In an embodiment, an inner lumen wall 112 or an outer lumen wall 102 can include a plastic, polymer, urethane, polyurethane, elastomer, rubber, silicone rubber, combinations thereof, or the like. In an embodiment, an inner lumen wall 112 can include a silicone rubber, rubber, or elastomer. In an embodiment, an outer lumen wall 102 can include a plastic, polymer, urethane, or polyurethane. In an embodiment, one or both of the inner lumen 104 and the outer lumen 114 can define a uniform lumen diameter between a proximal end 116 and a distal tip 118 of the catheter 100. In an embodiment, one or both of the inner lumen 104 and the outer lumen 114 can define a different wall thickness and/or different lumen diameters between a proximal end 116 and a distal tip 118 of the catheter 100, as described in more detail herein.

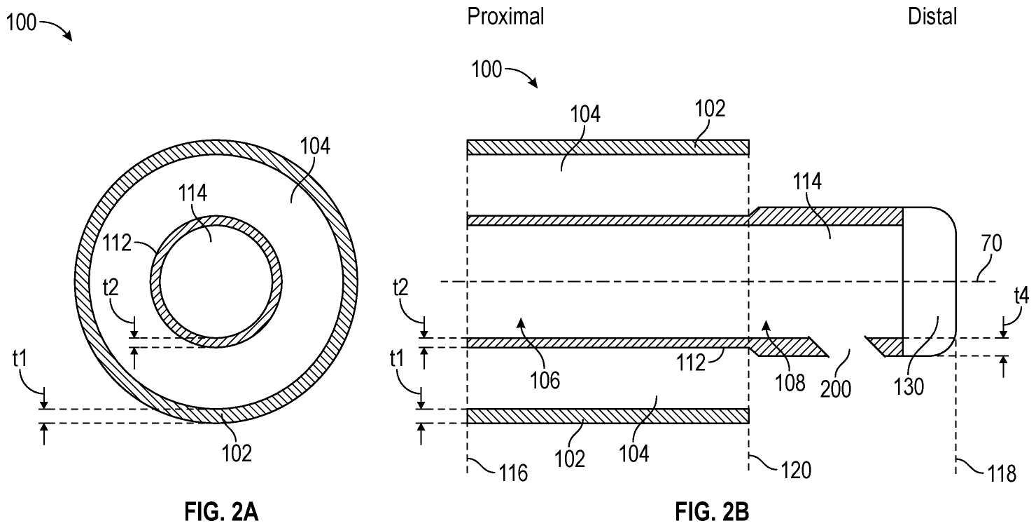

FIGS. 2A-2B show an embodiment of a catheter 100. FIG. 2A shows a lateral cross-section view of the catheter 100, and FIG. 2B shows a longitudinal cross-section view of the catheter 100. In an embodiment, a wall 112 of the inner lumen 114 can define a first wall thickness (t1) extending along a first portion, and a second wall thickness (t4) extending along a second portion. As shown, a proximal portion of the inner lumen wall 112 can define the first wall thickness (t1) and a distal portion of the inner lumen wall 112 can define the second wall thickness (t4). In an embodiment, the second wall thickness (t4) can be thicker than the first wall thickness (t1). However, it will be appreciated that other configurations, relative positions, and relative wall thicknesses of either the inner lumen wall 112 or the outer lumen wall 102 are also contemplated to fall within the scope of the present invention.

In an embodiment, as shown in FIG. 2B, a distal tip 118 of the catheter 100, i.e. a distal tip of one or both of the inner lumen 114 and the outer lumen 104, can include a closed distal end 130, defining an atraumatic tip. In an embodiment, one or both of the inner lumen 114 and the outer lumen 104 can include a valve 200 configured to provide fluid communication with a surrounding vasculature. For example, as shown in FIG. 2B, a distal tip of the inner lumen 114 can include a closed distal end 130 and can include a valve 200. Exemplary valves 200 can include a slit valve, flap valve, or the like.

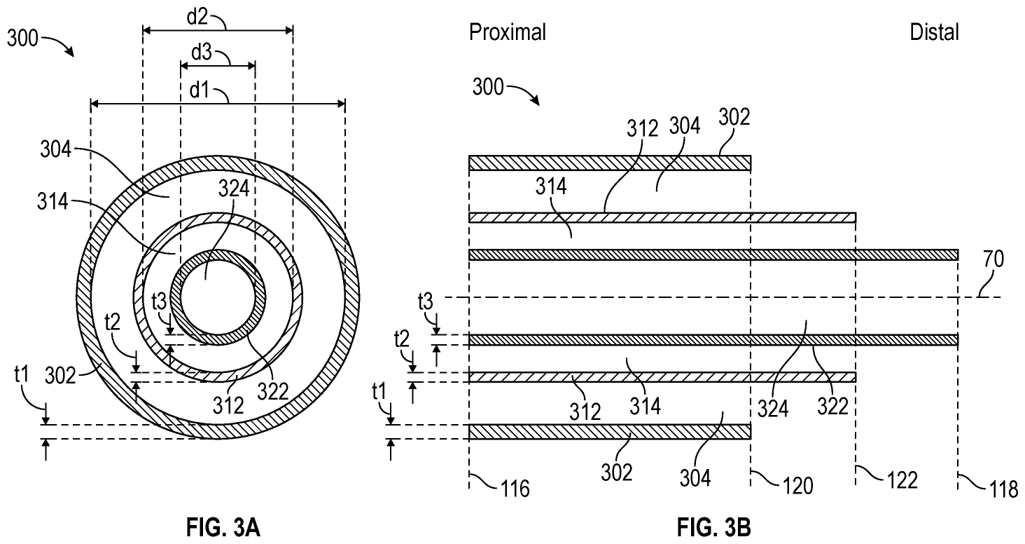

In an embodiment, as shown in FIGS. 3A-3B, a coaxial catheter 300 can include three or more lumen arranged coaxially, as described herein. FIG. 3A shows a lateral cross-sectional view of the catheter 300, and FIG. 3B shows a longitudinal cross-sectional view of the catheter 300. A triple-lumen catheter 300 includes a first wall 302 defining a first lumen 304, a second wall 312 defining a second lumen 314, and a third wall 322 defining a third lumen 324. As shown in FIG. 3B, one or more of the first lumen 304, the second lumen 314, and third lumen 324 can extend from the proximal end 116 of the catheter 300 to a distal tip 118.

In an embodiment, one or more of the first lumen 304 or the second lumen 314 can terminate a point that is proximal of the distal tip 118 of the catheter 300. In an embodiment, the first lumen 304 can terminate at a first distal tip 120 and the second lumen 314 can terminate at a second distal tip 122. One or more of the first distal tip 120 and the second distal tip 122 can be at the same or at different longitudinal positions along a length of the catheter 300. In an embodiment, the first lumen 304 can terminate at a first distal tip 120 in a lower arm target location of a patient, a second lumen 314 can terminate at a second distal tip 122 in the upper arm target location of the patient. The third lumen 324 can terminate at a distal tip 118 of the catheter 300 in the SVC target location. The relatively shorter lumens of the first lumen 304 and second lumen 314 may advantageously allow for higher infusion rates.

Since a pressure drop along a tube is proportional to its length, pressures exerted on the second lumen 314 distal to the distal tip 120 of the first lumen 304 can be lower than the proximal pressures, as described herein. In an embodiment, a fluid pressure within the second lumen 314 can support the wall 322 of the third lumen 324, as described herein. Similarly, a fluid pressure within the first lumen 304 can support the walls 312, 322 of one or both of the second lumen 314 and the third lumen 324, as described herein. In an embodiment, wall 312 of the second lumen 314 can support the wall 322 of the third lumen 324, as described herein. Similarly, the wall 302 of the first lumen 304 can support the walls 312, 322 of one or both of the second lumen 314 and the third lumen 324, as described herein.

In an embodiment, a wall 302 of the first lumen 304 can define a first thickness (t1), a wall 312 of the second lumen 314 can define a second wall thickness (t2), and a wall 322 of the third lumen 324 can define a third wall thickness (t3). In an embodiment, the first wall thickness (t1) can be greater than one or both of a second wall thickness (t2) and a third wall thickness (t3). In an embodiment, one or more of the first wall thickness (t1), second wall thickness (t2) and a third wall thickness (t3) can define a uniform wall thickness between a proximal end 116 and a distal tip 118 of the catheter 100. In an embodiment, one or more of the first wall thickness (t1), second wall thickness (t2) and a third wall thickness (t3) can define a different wall thickness between a proximal end 116 and a distal tip 118 of the catheter 100. These and other combinations of relative wall thicknesses (t1, t2, t3) are contemplated to fall within the scope of the present invention.

In an embodiment, the first lumen 304 can define a first lumen diameter (d1), a second lumen 314 can define a second lumen diameter (d2), and third lumen 324 can define a third lumen diameter (d3). In an embodiment, one or more of the first lumen 304, second lumen 314, and third lumen 324 (i.e. d1, d2, or d3) can define a uniform lumen diameter along a longitudinal length thereof. In an embodiment, one or more of the first lumen 304, second lumen 314, and third lumen 324 (i.e. d1, d2, or d3) can define a different lumen diameter along a longitudinal length thereof.

In an embodiment, one or more of the first wall 302, second wall 312, and third wall 324 can be formed of the same material or of different, displaying different mechanical properties. For example, one of the second wall 312 or the third wall 322 can be formed of a softer material, a more compliant material, or a material having a lower modulus of elasticity (i.e. easier to elastically deform), or combinations thereof, relative to the first wall 302. These and other numbers or combinations of wall thickness, lumen diameter, or materials, or combinations thereof, are contemplated to fall within the scope of the present invention.

FIGS. 4A-4B shows various cross-section views of a proximal end of a coaxial catheter 100, as described herein. FIG. 4A shows a lateral cross-sectional view of the co-axial catheter 100. FIG. 4B shows a longitudinal cross-sectional view of a co-axial catheter 100. In an embodiment, a proximal end 116 of the catheter 100 can include a bifurcation hub (“hub”) 150 and can include one or more extension legs, 160, 162 configured to provide fluid communication with one or more lumen of the catheter 100. For example, a first extension leg 160 can provide fluid communication with the outer lumen 104, and a second extension leg 162 can provide fluid communication with the inner lumen 114.

As will be appreciated, additional extension legs can provide fluid communication with one or more additional lumen, e.g. a second lumen 314, of the catheter 100. In an embodiment, the extension legs 160, 162 may be of different lengths to accommodate different distal termination points as shown in FIGS. 1A-1B. Further details regarding the bifurcation hub 150 of a coaxial catheter can be found, for example, in U.S. Pat. No. 7,896,853, which is incorporated by reference in its entirety into this application. Advantageously, having a catheter system that not only provides for efficient infusions, but ensures that the incidents of the opposite lumen reflux are precluded, advantageously reduces a risk of infusions of incorrect amounts of medications.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A multi-lumen catheter, comprising:
an outer wall defining an outer lumen having an outer lumen diameter and extending along a central longitudinal axis; and
an inner wall defining an inner lumen extending co-axially with the outer lumen and transitionable between a first position and a second position, the inner lumen in the first position having a first inner lumen diameter, and the inner lumen in the second position having a second inner lumen diameter larger than the first inner lumen diameter.

2. The multi-lumen catheter according to claim 1, wherein the inner wall is elastically deformable radially outwards from the first position to the second position.

3. The multi-lumen catheter according to claim 1, wherein one of the first inner diameter lumen or the second inner lumen diameter is equal to or less than the outer lumen diameter.

4. The multi-lumen catheter according to claim 1, wherein an outer surface of the inner wall contacts the inner surface of the outer wall when the inner lumen is in the second position.

5. The multi-lumen catheter according to claim 1, wherein the outer wall is formed of a first material, and the inner wall is formed of a second material, different from the first material.

6. The multi-lumen catheter according to claim 5, wherein the second material is more compliant, provides a lower durometer, is softer, or provides a lower modulus of elasticity relative to the first material.

7. The multi-lumen catheter according to claim 5, wherein a first material of the outer wall includes a plastic, polymer, urethane, or polyurethane, and a second material of the inner wall includes one of silicone rubber, rubber, or elastomer.

8. The multi-lumen catheter according to claim 1, wherein the inner wall in second position impinges on a surface of the outer wall, the outer wall preventing further radial expansion of the inner wall.

9. The multi-lumen catheter according to claim 1, wherein the outer wall defines a first thickness and the inner wall defines a second thickness, less than the first thickness.

10. The multi-lumen catheter according to claim 1, wherein the outer wall defines a first thickness and the inner wall defines a second thickness, one or both of the first thickness and the second thickness defines a uniform thickness along a longitudinal length of the catheter.

11. The multi-lumen catheter according to claim 1, wherein the outer wall defines a first thickness and the inner wall defines a second thickness, one or both of the first thickness and the second thickness defines a change in thickness along a longitudinal length of the catheter.

12. The multi-lumen catheter according to claim 1, wherein one of the first inner lumen diameter or the outer lumen diameter defines a uniform diameter along a longitudinal length of the catheter.

13. The multi-lumen catheter according to claim 1, wherein one of the first inner lumen diameter or the outer lumen diameter defines a change diameter along a longitudinal length of the catheter.

14. The multi-lumen catheter according to claim 1, wherein the inner lumen defines a first longitudinal length, and the outer lumen defines a second longitudinal length, the second longitudinal length being less than the first longitudinal length.

15. The multi-lumen catheter according to claim 1, wherein a distal tip of the outer lumen is configured to terminate in an upper arm portion of a patient, and a distal tip of the inner lumen is configured to terminate in a superior vena cava.

16. The multi-lumen catheter according to claim 1, further including a bifurcation hub, a first extension leg in fluid communication with the inner lumen, and a second extension leg in fluid communication with the outer lumen.

17. The multi-lumen catheter according to claim 1, wherein one of the inner lumen or the outer lumen includes a closed distal tip and a valve.

18. A catheter system, comprising:
a first wall defining a first lumen extending along a central longitudinal axis;
a second wall defining a second lumen extending co-axially with the first lumen along the central longitudinal axis, the first wall encircling the second wall;
a third wall defining a third lumen extending co-axially with the first lumen and the second lumen along the central longitudinal axis, the first wall and the second wall encircling the third wall, one of the second wall or the third wall is elastically deformable radially outwards.

19. The catheter system according to claim 18, wherein one or both of the second lumen and the third lumen define a lumen diameter is equal to or less than a lumen diameter of the first lumen.

20. The catheter system according to claim 18, wherein the third wall is configured to elastically deform radially outwards until an outer surface of the third wall impinges on the inner surface of the second wall.

21. The catheter system according to claim 18, wherein the second wall is configured to elastically deform radially outwards until an outer surface of the second wall impinges on the inner surface of the first wall.

22. The catheter system according to claim 18, wherein the first wall is configured to prevent radial outward expansion.

23. The catheter system according to claim 18, wherein the first wall is formed of a first material, and one or both of the second wall and the third wall is formed of a second material, different from the first material.

24. The catheter system according to claim 23, wherein the third wall is formed of a third material different from the first material and the second material.

25. The catheter system according to claim 24, wherein one or both of the second material and the third material is more compliant, provides a lower durometer, is softer, or provides a lower modulus of elasticity relative to the first material.

26. The catheter system according to claim 24, wherein the first material includes a plastic, polymer, urethane, or polyurethane, and one or both of the second material and the third material includes one of silicone rubber, rubber, or elastomer.

27. The catheter system according to claim 18, wherein the first wall, second wall and third wall define the same wall thickness.

28. The catheter system according to claim 18, wherein one of the first wall, second wall, or third wall define a different wall thickness.

29. The catheter system according to claim 18, wherein the first wall, second wall and third wall define a uniform wall thickness along a longitudinal length of the catheter.

30. The catheter system according to claim 18, wherein one of the first wall, second wall, or third wall define different wall thicknesses along a longitudinal length of the catheter.

31. The catheter system according to claim 18, wherein the first lumen, second lumen and third lumen define a uniform lumen diameter along a longitudinal length of the catheter.

32. The catheter system according to claim 18, wherein one of the first lumen, second lumen, or third lumen define different lumen diameters along a longitudinal length of the catheter.

33. The catheter system according to claim 18, wherein the first lumen, second lumen and third lumen define the same longitudinal length between a proximal end of the catheter and a distal tip of the catheter.

34. The catheter system according to claim 18, wherein one of the second lumen or third lumen terminate at a point proximal of a distal tip of the catheter.

35-40. (canceled)
